(19) United States
(12) Patent Application Publication
(10) Pub. No.: US 2010/0317699 A1
(43) Pub. Date: Dec. 16, 2010

(54) USE OF HEDGEHOG AGONISTS IN THE TREATMENT OF MUSCULOSKELETAL-RELATED DISORDERS

(75) Inventors: Dustin Donald Armstrong, Cambridge, MA (US); Shilpa Kadam, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG, Basel (CH)

(21) Appl. No.: 12/864,580

(22) PCT Filed: Jan. 27, 2009
(86) PCT No.: PCT/EP09/50868

§ 371 Date: Jul. 26, 2010

Related U.S. Application Data
(60) Provisional application No. 61/024,423, filed on Jan. 29, 2008.

(51) Int. Cl. A61K 31/4436; A61P 21/00; C12Q 1/02

(52) U.S. Cl. 514/337; 435/29

(57) ABSTRACT

The invention provides methods for the diagnosis and treatment of musculoskeletal disorders relating to the Hedgehog pathway, including but not limited to muscular dystrophy (e.g., Duchenne Muscular Dystrophy) using agents that agonize Sonic Hedgehog (shh), and thereby, the Hedgehog signaling pathway. Said agonizing agents include, e.g., the compounds of the invention (e.g., a compound of Formula I). The invention also provides methods of screening for agents that are capable of increasing the proliferation of muscle and/or muscle precursor cells.

### BACKGROUND OF THE INVENTION

Hedgehog (Hh) signaling was first identified in Drosophila as an important regulatory mechanism for embryonic pattern formation, or the process by which embryonic cells form ordered spatial arrangements of differentiated tissues. (Nusslein-Volhard et al. (1980) Nature 287, 795-801) In mammalian cells, three Hedgehog genes, Sonic Hedgehog (Shh), India Hedgehog (Ihh) and Desert Hedgehog (Dhh), have been identified. Hedgehog genes encode secreted proteins, which undergo post-translational modifications, including autocatalytic cleavage and lipid modification (palmitoylation) at the N-terminus and cholesterol modification of the C-terminus.

The lipid-modified N-terminal Hedgehog protein triggers the signaling activity of the protein pathway, and cell to cell communication is engendered by the dispatch of soluble Hedgehog protein from a signaling cell and receipt by a responding cell. In responding cells, the 12-pass transmembrane receptor Patched (Ptch) acts as negative regulator of Hh signaling and the 7-pass transmembrane protein Smoothened (Smo) acts as a positive regulator of Hh signaling. At resting state, free Ptch (i.e., unbound by Hh) substoichiometrically suppresses pathway activity induced by Smo (Taipale et al. (2002) Nature 418: 892); upon binding ligand Hh protein, however, repression of Smo is relieved, and the resulting signaling cascade leads to the activation and nuclear translocation of Gli transcription factors (Gli1, Gli2 and Gli3).

Downstream target genes of Hh signaling transcription include Wnts, TGFβ, and Ptc and Gli1, which are elements of the positive and negative regulatory feedback loop. Several cell-cycle and proliferation regulatory genes, such as c-myc, cyclin D and E are also among the target genes of Hh signaling.

Shh signaling is critical for embryonic development and particularly necessary for initiation of myogenesis and expansion of muscle progenitor cells. (Duprez, D. et al. (1998) Development 125, 495-505) (Cossu, G. et al. (1999) Embo J 18, 6867-72). Though less established, recent evidence indicates that Shh signaling is recapitulated in adult muscle following ischemia or muscle damage. (Pola, R. et al. (2003) Circulation 108, 479-85) Meanwhile, recombinant Shh protein (rShh) has been demonstrated to promote proliferation of muscle satellite cells (SC) isolated from both mouse and chick skeletal muscle. (Koleva, M. et al. (2005) Cell Mol Life Sci 62, 1863-70) (Elia, D. (2007) Biochim Biophys Acta 1773, 1438-46). SCs are the best-characterized resident stem cells in skeletal muscle, and they provide a means for intrinsic regeneration of skeletal muscle tissue. (Collins, C. A. et al. (2005) Cell Cycle 4, 1338-41) (Tajbakhsh, S. (2005) Exp Cell Res 306, 364-72). However, in cases of injury, genetic disease, or aging, this regenerative capacity is reduced. Sonic hedgehog (Shh) signaling is critical for precursor cell expansion during embryonic muscle development and becomes reactivated in adults following muscle damage.

Normally satellite cells remain quiescent at the edge of muscle fibers between the sarcolemma and basal lamina, but can be activated in response to muscle stimulation such as exercise or trauma. (Bornemann, A. et al. (1999) Neuropediatrics 30, 167-75) In humans, both the number and activating potential of satellite cells are thought to decrease with age. (Sajko, S. et al. (2004) J Histochem Cytochem 52, 179-85) Furthermore, patients with inherited diseases such as muscular dystrophy can suffer from regenerative failure, indicating that satellite cell replacement is not unlimited.

These type of myopathies may eventually be treated with cell-based therapies; however, a pharmacological treatment to enhance the proliferation of endogenous stem cells would be of significant therapeutic value. Agonists of Hh signaling pathways are thought to have therapeutically utility as treatments for muscle injury or disease, and as such, are of interest to researchers in the field and the broader scientific community. As there are presently no compounds capable of activating endogenous muscle satellite cells, identification of Shh agonizing compounds capable of acting in an analogous fashion to recombinant Shh protein are desirable for the treatment for myopathies and musculoskeletal disorders.

### SUMMARY OF THE INVENTION

The present invention relates generally to the diagnosis and treatment of musculoskeletal disorders relating to the Hedgehog pathway, including but not limited to muscular dystrophy (e.g., Duchenne Muscular Dystrophy) using agents that agonize Sonic Hedgehog (shh), and thereby, the Hedgehog signaling pathway. Said agonizing agents include, e.g., the compounds of the invention (e.g., a compound of Formula I). The methods and compounds of the present invention relate to agonizing the Hedgehog signaling pathway, e.g., by activating the Smo receptor independent of the Shh ligand, and comprise contacting the cell with a compounds of the invention (e.g., a compound of Formula I) in a sufficient amount to agonize a normal Shh activity, antagonize a normal Ptc activity, or agonize smoothened activity (e.g., to reverse or control the aberrant growth state).

One aspect of the present invention makes available methods employing compounds for agonizing Hedgehog (ligand)-independent pathway activation. In certain embodiments, the present methods can be used to counteract the phenotypic effects of unwanted inhibition of a Hedgehog pathway, such as resulting from Hedgehog loss-of-function, Ptc gain-of-function, or smoothened loss-of-function mutations. For instance, the subject method can involve contacting a cell (in vitro or in vivo) with a Shh agonist, such as a compound of the invention (e.g., a compound of Formula I (e.g., Shh-Ag)) or other small molecule in an amount sufficient to agonize a Hedgehog-independent activation pathway.

The compounds of the invention, as further described below, include small molecule agonists of Hedgehog (e.g., Sonic Hedgehog) synthesis, expression, production, stabilization, phosphorylation, relocation within the cell, and/or activity. The compounds of the invention include but are not limited to compounds of Formula I.

This invention provides a method for determining whether an agent, known to upregulate the sonic hedgehog pathway, increases primary myoblast proliferation (e.g., in a PI3K/Akt-dependent manner) comprising: (i) administering the agent to a non-human subject; and (ii) determining whether the resulting primary myoblast proliferation in the subject is greater than that in a subject to which the agent was not administered, thereby determining whether the agent increases primary myoblast proliferation.

This invention provides a method for determining whether an agent, known to upregulate the sonic hedgehog pathway, promotes satellite cell (SC) proliferation comprising: (i) administering the agent to a non-human subject; and (ii) determining whether the resulting satellite cell (SC) proliferation in the subject is greater than that in a subject to which the agent was not administered, thereby determining whether the agent promotes satellite cell (SC) proliferation.

This invention further provides a method for treating musculoskeletal disorders by administering to an afflicted subject a therapeutically effective amount of an agent known to agonize or otherwise upregulate the sonic hedgehog pathway and, determined to have the ability to increase cellular proliferation (e.g., of primary myoblasts and/or satellite cells (SCs)), wherein such ability is determined by a method comprising (i) administering the agent to a non-human subject, and (ii) determining whether the resulting cellular proliferation in the subject is greater than that in a subject to which the agent was not administered.

This invention further provides a method for inhibiting the onset of musculoskeletal disorders by administering to a subject in need thereof a prophylactically effective amount of an agent known to agonize or otherwise upregulate the sonic hedgehog pathway, and determined as having the ability to increase cellular proliferation (e.g., of primary myoblasts and/or satellite cells (SCs)), wherein such ability is determined by a method comprising (i) administering the agent to a non-human subject, and (ii) determining whether the resulting increase in cellular proliferation in the subject is greater than that in a subject to which the agent was not administered.

This invention further provides a composition comprising (a) a pharmaceutically acceptable carrier, and (b) an agent known to upregulate the sonic hedgehog pathway, and determined as having the ability to increase cellular proliferation (e.g., of primary myoblasts and/or satellite cells (SCs)), wherein such ability is determined by a method comprising (i) administering the agent to a non-human subject, and (ii) determining whether the resulting increase in cellular proliferation (e.g., of primary myoblasts and/or satellite cells (SCs)) in the subject is greater than that in a subject to which the agent was not administered.

This invention further provides an article of manufacture comprising a packaging material having therein an agent known to upregulate the sonic hedgehog pathway, and determined as having the ability to increase cellular proliferation (e.g., of primary myoblasts and/or satellite cells (SCs)), and a label indicating a use of the agent for inhibiting the onset of a musculoskeletal disorder in a subject, wherein such ability is determined by a method comprising (i) administering the agent to a non-human subject, and (ii) determining whether the resulting increase in cellular proliferation (e.g., of primary myoblasts and/or satellite cells (SCs)) in the subject is greater than that in a subject to which the agent was not administered.

This invention further provides an article of manufacture comprising a packaging material having therein an agent known to upregulate the sonic hedgehog pathway, and determined as having the ability to increase cellular proliferation (e.g., of primary myoblasts and/or satellite cells (SCs)), and a label indicating a use of the agent for treating a musculoskeletal disorder in a subject, wherein such ability is determined by a method comprising (i) administering the agent to a non-human subject, and (ii) determining whether the resulting increase in cellular proliferation (e.g., of primary myoblasts and/or satellite cells (SCs)) in the subject is greater than that in a subject to which the agent was not administered.

The methods of the present invention may be used to regulate proliferation of cells (e.g., of primary myoblasts and/or of satellite cells (SCs)) in vitro and/or in vivo, e.g., in the formation of new or regenerated muscle tissues. In another particular embodiment, contacting the cell with—or introducing into the cell—a compound of the invention (e.g., a compound of Formula I) results in promotion of cellular proliferation and recovery from musculoskeletal injury or disorder. Thus, another particular embodiment provides methods for agonizing the Hh pathway by employing compounds of the invention (e.g., a compound of Formula I) in an injured or diseased muscle cell.

### BRIEF DESCRIPTION OF THE FIGURES

FIG. 1 demonstrates that Shh-Ag treatment induces Gli1 expression and promotes myoblast and satellite cell proliferation. As seen in FIG. 1A, Gli1 mRNA is upregulated in the sonic-hedgehog sensitive leydig cell-line, TM3, primary mouse myoblasts (PM), C2C12 myoblasts, and isolated satellite cells (SC) following Shh-Ag (10 μM) treatment for 24 hours. Graph represents mean values±standard deviation and are expressed relative to vehicle treated=1. *P<0.05. As seen in FIG. 1B, Shh-Ag treatment (24 hrs) promotes proliferation of primary myoblasts, increasing the percentage of cells in G₂/M phase of the cell cycle. Both an example and a graphic summary of FACS analysis following PI staining are shown. *P<0.05. As seen in FIG. 1C, treatment with Shh-Ag for 48 hours induces proliferation of isolated SCs, increasing both total cell number and percent of BrdU incorporating cells. *P<0.05.

FIG. 2A depicts the blocking action of LY29002 (1 μM), a phosphatidylinositol 3-kinase/Akt pathway inhibitor, on Shh-Ag induced myoblast proliferation. FIG. 2B shows that the increase in Gli1 and Cyclin D1 protein levels following Shh-Ag or recombinant sonic hedgehog protein (rSHH) treatment (1 μg/mL) are completely blocked by LY29002-mediated PI3K/AKT inhibition. Values represent mean densitometric scores expressed relative to vehicle=100. *P<0.05.

FIG. 3 is a graphic depiction of the design of in vivo experiments, further detailed herein. 6 to 8-week old wildtype (WT), DMD^{MDX}, or WT cardiotoxin-injured mice (Ctx-inj) received two consecutive bilateral IM injections of either vehicle (10% DMSO/PBS), or Shh-Ag (500 μM) into tibialis anterior (TA) or gastrocnemius (GA) muscles, followed by an IP injection of BrdU. Mice were sacrificed and muscles were then collected 3 or 7 days following ctx-injury for immunostaining (TA) or FACS analysis (GA); n=4 for all groups.

FIG. 4 demonstrates that Shh-Ag injection improves regeneration following cardiotoxin-injury. To quantify Shh-Ag activated satellite cell proliferation, satellite cells were isolated from treated muscle (GA) and examined by PACS analysis for BrdU expression. While the total percentage of satellite cells increases only slightly with Shh-Ag treatment, the percent of BrdU+ satellite cells almost doubles (4.8 to 10.6%, respectively). Graph represents the mean percentage of cells out of the entire single-cell preparation±standard deviation. *P<0.05.

### DETAILED DESCRIPTION OF THE INVENTION

The present invention relates to the discovery that signal transduction pathways regulated by Shh, patched (ptc), gli and/or smoothened can be modulated, at least in part, by small molecules. While not wishing to be bound by any particular theory, the activation of a patched-smoothened pathway through alteration of cell-surface associations (such as complexes) may be the mechanism by which these agents act.

The Hh pathway is believed to be negatively regulated by association of patched and smoothened, such as in the form of protein complexes, which association is disrupted by the binding of hedgehog to patched. Accordingly, the ability of these agents to activate the hedgehog pathway may be due to the ability of such molecules to interact with or bind to patched or smoothened, to otherwise disrupt the association of smoothened with patched, or at least to promote the ability of those proteins to activate a hedgehog, ptc, and/or smoothened-mediated signal transduction pathway. This mode of action, e.g., modulation of a smoothened-dependent pathway, is to be distinguished from compounds which modulate the hedgehog pathway by directly activating the cAMP pathway, e.g., by binding to or interacting with PKA, adenylate cyclase, cAMP phosphodiesterase, etc.

In certain embodiments, hedgehog agonists disclosed herein modulate hedgehog activity in the absence of hedgehog protein itself, e.g., the compounds mimic the activity of hedgehog, rather than merely supplement or increase the activity of hedgehog protein, e.g., by promoting hedgehog binding to patched. These compounds are referred to herein as hedgehog-independent agonists and alone can mimic the phenotype or effect resulting from hedgehog treatment. In other embodiments, the present compounds enhance the activity of hedgehog protein, and require the presence or addition of hedgehog protein to observe the phenotype or effect resulting from hedgehog induction. Such hedgehog-dependent agonists may be used in therapeutic preparations or treatments which include hedgehog protein, or may be used to increase the activity of hedgehog protein naturally produced by the cells or tissue to be treated with the agonist. The hedgehog agonists disclosed herein may induce dissociation of a patched-smoothened complex or disrupt interactions between patched and smoothened, such as by binding to patched or to smoothened, thereby activating the hedgehog pathway. In certain embodiments, the compositions and methods of the present invention employ a compound which acts on one or more components of the extracellular membrane of a target cell.

In certain embodiments, hedgehog agonists useful in the present induce hedgehog-dependent transcriptional regulation, such as expression of the gli1 or ptc genes. Such agonists can thus induce or increase the hedgehog-dependent pathway activation resulting from, for example, increased levels of hedgehog protein.

It is, therefore, specifically contemplated that these small molecules which modulate aspects of hedgehog, ptc, or smoothened signal transduction activity will likewise be capable of promoting proliferation (or other biological consequences) in cells having a functional ptc-smo pathway. In preferred embodiments, the subject agonists are organic molecules having a molecular weight less than 2500 amu, more preferably less than 1500 amu, and even more preferably less than 750 amu, and are capable of inducing or augmenting at least some of the biological activities of hedgehog proteins, preferably specifically in target cells. Activation of the hedgehog pathway by a hedgehog agonist may be quantified, for example, by detecting the increase in ptc or gli-1 transcription in the presence of the agonist relative to a control in the absence of agonist. For example, an increase of at least 5%, at least 10%, at least 20%, or even at least 50% may be indicative of hedgehog pathway activation by a test compound.

In certain embodiments, a compound useful in the present invention, such as described above, may have an EC 50 for inducing or augmenting one or more Hh activities (such as upregulation of ptc or gli expression) of less than about 1000 nM, less than about 100 nM, less than about 10 nM, or even less than about 1 nM. The coding sequences for exemplary human Gli genes include, for example, the Gli-1 gene sequence of GenBank accession X07384 and the Gli-2 gene sequence of GenBank accession AB007298. The level of gli or ptc expression can be determined, for example, by measuring the level of mRNA (transcription) or the level of protein (translation).

In another aspect, the present invention provides pharmaceutical preparations comprising, as an active ingredient, a hedgehog agonist, ptc antagonist, or smoothened agonist such as described herein, formulated in an amount sufficient to promote, in vivo, proliferation or other biological consequences.

The present invention relates to compounds of the invention, including compounds, of the formula (I):

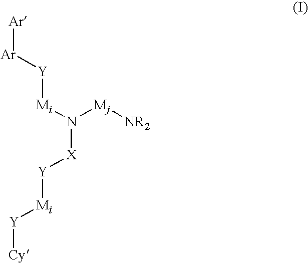

wherein, as valence and stability permit,

Ar and Ar′ independently represent substituted or unsubstituted aryl or heteroaryl rings;

Y, independently for each occurrence, is absent or represent s—N(R)—, —O—, —S—, or —Se—;

X is selected from —C(═O)—, —C(═S)—, —S(O₂)—, —S(O)—, —P(═O)(OR)—, and a methylene group optionally substituted with 1-2 groups such as lower alkyl, alkenyl, or alkynyl groups;

M represents, independently for each occurrence, a substituted or un-substituted methylene group, such a s—CH2-, —CHF—, —CHOH—, —CH(Me)—, —C(═O)—, etc., or two M taken together represent substituted or unsubstituted ethene or ethyne, wherein some or all occurrences of M in Mⱼ form all or part of a cyclic structure;

R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;

Cy′ represents a substituted or unsubstituted aryl, heterocyclyl, heteroaryl, or cycloalkyl, including polycyclic groups;

j represents, independently for each occurrence, an integer from 0 to 10, preferably from 2 to 7; and

i represents, independently for each occurrence, an integer from 0 to 5, preferably from 0 to 2.

In certain embodiments, M represents, independently for each occurrence, a substituted or unsubstituted methylene group, such as —CH2-, —CHF—, —CHOH—, —CH(Me)—, —C(═O)—, etc.

In certain embodiments, Ar and Ar′ represent phenyl rings, e.g., unsubstituted or substituted with one or more groups including heteroatoms such as O, N, and S. In certain embodiments, at least one of Ar and Ar′ represents a phenyl ring. In certain embodiments, at least one of Ar and Ar′ represents a heteroaryl ring, e.g., a pyridyl, thiazolyl, thienyl, pyrimidyl, etc. In certain embodiments, Y and Ar′ are attached to Ar in a meta and/or 1,3-relationship.

In certain embodiments, Y is absent from all positions. In embodiments wherein Y is present in a position, i preferably represents an integer from 1-2 in an adjacent M, if i=0 would result in two occurrences of Y being directly attached, or an occurrence of Y being directly attached to N or NR₂.

In certain embodiments, Cy′ is a substituted or unsubstituted aryl or heteroaryl. In certain embodiments, Cy′ is directly attached to X. In certain embodiments, Cy′ is a substituted or unsubstituted bicyclic or heteroaryl ring, preferably both bicyclic and heteroaryl, such as benzothiophene, benzofuran, benzopyrrole, benzopyridine, etc. In certain embodiments, Cy′ is a monocyclic aryl or heteroaryl ring substituted at least with a substituted or unsubstituted aryl or heteroaryl ring, i.e., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, X is selected from —C(O)—, —C(═S)—, and —S(O₂)—.

In certain embodiments, R represents H or lower alkyl, e.g., H or Me.

In certain embodiments, NR₂ represents a primary amine or a secondary or tertiary amine substituted with one or two lower alkyl groups, aryl groups, or aralkyl groups, respectively, preferably a primary amine or secondary amine.

In certain embodiments, substituents on Ar or Ar′ are selected from halogen, lower alkyl, lower alkenyl, aryl, heteroaryl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above, wherein p and n, individually for each occurrence, represent integers from 0 to 10, preferably from 0 to 5.

As further described below, the compounds of the invention can be prepared as described in US patent publications US20050070578 and related family members, the contents of all of which are herein incorporated by reference.

In certain embodiments, the Shh-Ag is a compound of the invention (e.g., a compound of Formula I), as follows:

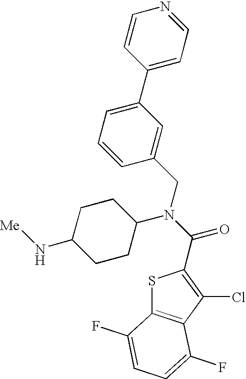

In the present description, the term “treatment” includes both prophylactic or preventive treatment as well as curative or disease suppressive treatment, including treatment of patients at risk for a disorder of the invention (e.g., a musculoskeletal disorder (e.g., muscular dystrophy)) as well as ill patients. This term further includes the treatment for the delay of progression of the disease.

By “suppress and/or reverse,” e.g., a musculoskeletal disorder (e.g., muscular dystrophy), Applicants mean to abrogate said musculoskeletal disorder (e.g., muscular dystrophy), or to render said condition less severe than before or without the treatment.

“Cure” as used herein means to lead to the remission of the musculoskeletal disorder (e.g., muscular dystrophy) in a patient, or of ongoing episodes thereof, through treatment.

The terms “prophylaxis” or “prevention” means impeding the onset or recurrence of musculoskeletal disorders, e.g., muscular dystrophy.

“Treatment” or “treating” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted.

“Diagnosis” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

“Subject” or “patient” refers to a mammal, preferably a human, in need of treatment for a condition, disorder or disease.

“A compound(s) of the invention” as used herein includes but is not limited to compounds of Formula I. A compound of the invention includes the specifically listed compounds listed herein, including those listed in the Examples of the present application.

“Delay of progression” as used herein means that the administration of a compound of the invention (e.g., a compound of Formula I) to patients in a pre-stage or in an early phase of a musculoskeletal disorder (e.g., muscular dystrophy) prevents the disease from evolving further, or slows down the evolution of the disease in comparison to the evolution of the disease without administration of the active compound.

As used herein a “small organic molecule” is an organic compound (or organic compound complexed with an inorganic compound (e.g., metal)) that has a molecular weight of less than 3 kilodaltons, and preferably less than 1.5 kilodaltons.

As used herein a “reporter” gene is used interchangeably with the term “marker gene” and is a nucleic acid that is readily detectable and/or encodes a gene product that is readily detectable such as luciferase.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

“Analog” as used herein, refers to a small organic compound, a nucleotide, a protein, or a polypeptide that possesses similar or identical activity or function(s) as the compound, nucleotide, protein or polypeptide or compound having the desired activity and therapeutic effect of the present invention. (e.g., inhibition of tumor growth), but need not necessarily comprise a sequence or structure that is similar or identical to the sequence or structure of the preferred embodiment

“Derivative” refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

“Agonists” and “mimetics” may be agents that increase, facilitate, promote, or engender, signaling (e.g., Hh signaling) via a pathway and/or which prevent the formation of protein interactions and complexes.

“Musculoskeletal disorder(s)” as used herein includes bone diseases (e.g., metabolic bone diseases), bursitis, cartilage diseases, joint diseases, myotonias, neuromyotonia, cachexia and wasting, ischemia, Poland Syndrome, muscular dystrophies (e.g., Duchenne and Becker Muscular Dystrophies, and Limb Girdle Muscular Dystrophies), musculoskeletal abnormalities, myopathies (e.g., congenital myopathies, inflammatory myopathies, centronuclear myopathy, myotubular myopathy, and metabolic myopathies (e.g., glycogen storage diseases, lipid storage disorders)), osteoarthritis, osteochondritis, Osteogenesis Imperfecta, osteomyelitis, osteonecrosis, osteopetrosis, osteoporosis, and scoliosis. “Musculoskeletal disorder(s)” as used herein also includes muscle or skeletal injuries, as well as muscle or skeletal disorders relating to genetic disease and/or aging.

The term “alkyl” refers to straight or branched chain hydrocarbon groups having 1 to 20 carbon atoms, preferably lower alkyl of 1 to 7 carbon atoms. Exemplary alkyl groups include methyl, ethyl, propyl, isopropyl, n-butyl, t-butyl, isobutyl, pentyl, hexyl, isohexyl, heptyl, 4,4-dimethylpentyl, octyl and the like. Preferred is C₁-C₄-alkyl.

The term “lower” referred to herein in connection with organic radicals or compounds respectively generally defines, if not defined differently, such with up to and including 7, preferably up and including 4 and advantageously one or two carbon atoms. Such may be straight chain or branched.

The term “optionally substituted alkyl” refers to unsubstituted or substituted straight or branched chain hydrocarbon groups having 1 to 20 carbon atoms, preferably lower alkyl of 1 to 7 carbon atoms. Exemplary unsubstituted alkyl groups include methyl, ethyl, propyl, isopropyl, n-butyl, t-butyl, isobutyl, pentyl, hexyl, isohexyl, heptyl, 4,4-dimethylpentyl, octyl and the like.

The term “substituted alkyl” refers to alkyl groups substituted by one or more of the following groups: halo (such as F, Cl, Br and I), hydroxy, alkoxy, alkoxyalkoxy, aryloxy, cycloalkyl, alkanoyl, alkanoyloxy, amino, substituted amino, alkanoylamino, thiol, alkylthio, arylthio, alkylthiono, alkylsulfonyl, arylsulfonyl, heteroarylsulfonyl, aminosulfonyl, nitro, cyano, carboxy, carbamyl, alkoxycarbonyl, aryl, aralkoxy, guanidino, heterocyclyl (e.g., indolyl, imidazolyl, furyl, thienyl, thiazolyl, pyrrolidyl, pyridyl, pyrimidyl), and the like.

The term “lower alkyl” refers to those alkyl groups as described above having 1 to 7, preferably 1 to 4 carbon atoms. The term “halogen” or “halo” refers to fluorine, chlorine, bromine and iodine.

The term “alkoxy” or “alkyloxy” refers to alkyl-O—.

The term “aryl” or “ar”, refers to carbocyclic monocyclic or bicyclic aromatic hydrocarbon groups having 6 to 12 carbon atoms in the ring portion, such as phenyl, naphthyl, tetrahydronaphthyl, and biphenyl groups, each of which may optionally be substituted by one to four, e.g., one or two, substituents such as alkyl, halo, trifluoromethyl, hydroxy, alkoxy, alkanoyl, alkanoyloxy, amino, substituted amino, alkanoylamino, thiol, alkylthio, nitro, cyano, carboxy, carboxyalkyl, carbamyl, alkoxycarbonyl, alkylthiono, alkylsulfonyl, aminosulfonyl, and the like.

The term “aralkyl” refers to an aryl group linked to an alkyl group, such as benzyl.

The term “halogen” or “halo” refers to fluorine, chlorine, bromine and iodine.

The term “haloalkyl” refers to alkyl which mono- or polysubstituted by halo, such as trifluoromethoxy.

The term “alkylene” refers to a straight chain bridge of 1 to 6 carbon atoms connected by single bonds (e.g., —(CH₂)ₓ— wherein x is 1 to 6) which may be substituted with 1 to 3 lower alkyl groups.

The term “alkylene interrupted by O, S, N—(H, alkyl or aralkyl)” refers to a straight chain of 2 to 6 carbon atoms which is interrupted by O, S, N—(H, alkyl or aralkyl), such as (m)ethyleneoxy(m)ethylene, (m)ethylenethio(m)ethylene, or (m)ethyleneimino(m)ethylene.

The term “cycloalkyl” refers to cyclic hydrocarbon groups of 3 to 8 carbon atoms such as cyclopentyl, cyclohexyl or cycloheptyl.

The term “alkanoyloxy refers to alkyl-C(O)—O—.

The terms “alkylamino” and “dialkylamino” refer to (alkyl)NH— and (alkyl)₂N—, respectively.

The term “alkanoylamino” refers to alkyl-C(O)—NH—.

The term “alkylthio” refers to alkyl-S—.

The term “alkylthiono” refers to alkyl-S(O)—.

The term “alkylsulfonyl” refers to alkyl-S(O)₂—

The term “carbamyl” refers to —C(O)-amino or —C(O)-substituted amino. The term “alkoxycarbonyl” refers to alkyl-O—C(O)—.

The term “acyl” refers to alkanoyl, aroyl, heteroaryol, aryl-alkanoyl, heteroarylalkanoyl, and the like.

The term “heteroaryl” or “heteroar” refers to an aromatic heterocycle, for example monocyclic or bicyclic heterocyclic aryl, such as pyrrolyl, pyrazolyl, imidazolyl, oxazolyl, thiazolyl, isoxazolyl, thiazolyl, isothiazolyl, furyl, thienyl, pyridyl, pyrazinyl, pyrimidinyl, pyridazinyl, indolyl, benzothiazolyl, benzoxazolyl, benzothienyl, quinolinyl, isoquinolinyl, benzimidazolyl, benzofuryl, and the like, optionally substituted by one to four, e.g. one or two, substituents, such as lower alkyl, lower alkoxy or halo, the point of attachment of said heterocycle being at a carbon atom of the heterocyclic ring. Preferred heteroaryl residues are 1-methyl-2-pyrrolyl, 2-, 3-thienyl, 2-thiazolyl, 2-imidazolyl, 1-methyl-2-imidazolyl, 2-, 3-, 4-pyridyl, or 2-quinolyl.

The term “alkanoyl” refers, for example, to C₂-C₇-alkanoyl, especially C₂-C₅-alkanoyl, such as acetyl, propionyl or pivaloyl.

The term “aralkoxy” refers to an aryl group linked to an alkoxy group.

The term “arylsulfonyl” refers to aryl-SO₂—.

The term “aroyl” refers to aryl-CO—.

The term “carbonyl” is art-recognized and includes such moieties as can be represented by the general formula:

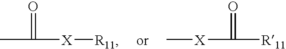

wherein X is a bond or represents an oxygen or a sulfur, and R₁₁ represents a hydrogen, an alkyl, an alkenyl, —(CH₂)ₘR8 or a pharmaceutically acceptable salt, R′₁₁ represents a hydrogen, an alkyl, an alkenyl or—(CHH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R₁₁′ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “heterocyclyl” refers to an optionally substituted, fully saturated or unsaturated, aromatic or nonaromatic cyclic group, for example, which is a 4 to 7 membered monocyclic, 7 to 11 membered bicyclic, or 10 to 15 membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized and the nitrogen heteroatoms may also optionally be quaternized. The heterocyclic group may be attached at any heteroatom or carbon atom.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

Exemplary bicyclic heterocyclic groups include indolyl, benzothiazolyl, benzoxazolyl, benzothienyl, quinuclidinyl, quinolinyl, tetrahydroisoquinolinyl, isoquinolinyl, benzimidazolyl, benzopyranyl, indolizinyl, benzofuryl, chromonyl, coumarinyl, enzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl) and the like.

Exemplary tricyclic heterocyclic groups include carbazolyl, benzidolyl, phenanthrolinyl, acridinyl, phenanthridinyl, xanthenyl and the like.

The term “heterocyclyl” also includes substituted heterocyclic groups. Substituted heterocyclic groups refer to heterocyclic groups substituted with 1, 2 or 3 of the following:

(a) alkyl;

(b) hydroxy (or protected hydroxy);

(c) halo;

(d) oxo (i.e. ═O);

(e) amino or substituted amino;

(f) alkoxy;

(g) cycloalkyl;

(h) carboxy;

(i) heterocyclooxy;

(j) alkoxycarbonyl, such as unsubstituted lower alkoxycarbonyl;

(k) carbamyl, alkylcarbamyl, arylcarbamyl, dialkylcarbamyl;

(l) mercapto;

(m) nitro;

(n) cyano;

(o) sulfonamido, sulfonamidoalkyl or sulfonamidodialkyl;

(p) aryl;

(q) alkylcarbonyloxy;

(r) arylcarbonyloxy;

(s) arylthio;

(t) aryloxy;

(u) alkylthio;

(v) formyl;

(w) arylalkyl; or

(x) aryl substituted with alkyl, cycloalkyl, alkoxy, hydroxy, amino, alkylamino, dialkylamino or halo.

The term “heterocyclooxy” denotes a heterocyclic group bonded through an oxygen bridge.

The term “heteroaryl” or “heteroar” refers to an aromatic heterocycle, for example monocyclic or bicyclic aryl, such as pyrrolyl, pyrazolyl, imidazolyl, oxazolyl, isoxazolyl, thiazolyl, isothiazolyl, furyl, thienyl, pyrazinyl, pyrimidinyl, pyridazinyl, indolyl, benzothiazolyl, benzoxazolyl, benzothienyl, quinolinyl, isoquinolinyl, benzimidazolyl, benzofuryl, and the like, optionally substituted by e.g., lower alkyl, lower alkoxy or halo.

The term “heteroarylsulfonyl” refers to heteroaryl-SO₂—

The term “heteroaroyl” refers to heteroaryl-CO—.

The term “acylamino” is art-recognized and refers to a moiety that can be represented by the general formula:

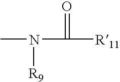

wherein R₉ represents a hydrogen, an alkyl, an alkenyl, —(CH₂)ₘ—R₈, or R₉ taken together with the N atom to which it is attached complete a heterocycle having from 4 to 8 atoms in the ring structure; R8 represents an aryl, a cycloalkyl, a cycloalkenyl, a heterocycle or a polycycle; and m is zero or an integer in the range of 1 to 8. R′₁₁ represents a hydrogen, an alkyl, an alkenyl or —(CH₂)ₘ—R₈.

The term “substituted amino” refers to amino mono- or, independently, disubstituted by alkyl, aralkyl, aryl, heteroaryl, cycloalkyl, cycloalkylalkyl, heteroaralkyl, or disubstituted by lower alkylene or lower alkylene interrupted by O, S, N—(H, alkyl, aralkyl) and the like.

Pharmaceutically acceptable salts of any acidic compounds of the invention are salts formed with bases, namely cationic salts such as alkali and alkaline earth metal salts, such as sodium, lithium, potassium, calcium, magnesium, as well as ammonium salts, such as ammonium, trimethylammonium, diethylammonium, and tris-(hydroxymethyl)-methylammonium salts.

Similarly, acid addition salts, such as of mineral acids, organic carboxylic, and organic sulfonic acids e.g., hydrochloric acid, methanesulfonic acid, maleic acid, are possible provided a basic group, such as amino or pyridyl, constitutes part of the structure.

Pharmaceutically acceptable salts of the compounds of the invention are particularly acid addition salts, such as of mineral acids, organic carboxylic, and organic sulfonic acids e.g., hydrochloric acid, methanesulfonic acid, maleic acid, and the like provided a basic group, such as pyridyl, constitutes part of the structure.

The compounds of the invention depending on the nature of the substituents, possess one or more asymmetric carbon atoms, and therefore exist as racemates and the (R) and (S) enantiomers thereof. All are within the scope of the invention. Preferred is the more active enantiomer typically assigned the S-configuration (at the carbon being the NR₆R₇ substituent).

The present invention relates to the discovery that agonizing the Hedgehog pathway can result in increasing the proliferation of primary myoblasts and satellite cells (SCs), e.g., by the compounds of the invention (e.g., a compound of Formula I). The present invention also relates to the discovery that agonizing pathways upstream of the Hedgehog pathway, such as the PI3K/Akt pathway, can result in increasing the proliferation of primary myoblasts and satellite cells (SCs), e.g., by the compounds of the invention (e.g., a compound of Formula I).

The present invention relates generally to the diagnosis and treatment of musculoskeletal disorders relating to the Hedgehog pathway, including but not limited to muscular dystrophy (e.g., Duchenne Muscular Dystrophy) using agents that agonize Sonic Hedgehog (shh), and thereby, the Hedgehog signaling pathway. Said agonizing agents include, e.g., the compounds of the invention (e.g., a compound of Formula I). The methods and compounds of the present invention relate to agonizing the Hedgehog signaling pathway, e.g., by activating the Smo receptor independent of the Shh ligand, and comprise contacting the cell with a compounds of the invention (e.g., a compound of Formula I) in a sufficient amount to agonize a normal Shh activity, antagonize a normal Ptc activity, or agonize smoothened activity (e.g., to reverse or control the aberrant growth state).

One aspect of the present invention makes available methods employing compounds for agonizing Hedgehog (ligand)-independent pathway activation. In certain embodiments, the present methods can be used to counteract the phenotypic effects of unwanted inhibition of a Hedgehog pathway, such as resulting from Hedgehog loss-of-function, Ptc gain-of-function, or smoothened loss-of-function mutations. For instance, the subject method can involve contacting a cell (in vitro or in vivo) with a Shh agonist, such as a compound of the invention (e.g., a compound of Formula I (e.g., Shh-Ag)) or other small molecule in an amount sufficient to agonize a Hedgehog-independent activation pathway.

The compounds of the invention, as further described below, include small molecule agonists of Hedgehog (e.g., Sonic Hedgehog) synthesis, expression, production, stabilization, phosphorylation, relocation within the cell, and/or activity. The compounds of the invention include but are not limited to compounds of Formula I.

This invention provides a method for determining whether an agent, known to upregulate the sonic hedgehog pathway, increases primary myoblast proliferation (e.g., in a PI3K/Akt-dependent manner) comprising: (i) administering the agent to a non-human subject; and (ii) determining whether the resulting primary myoblast proliferation in the subject is greater than that in a subject to which the agent was not administered, thereby determining whether the agent increases primary myoblast proliferation.

This invention provides a method for determining whether an agent, known to upregulate the sonic hedgehog pathway, promotes satellite cell (SC) proliferation comprising: (i) administering the agent to a non-human subject; and (ii) determining whether the resulting satellite cell (SC) proliferation in the subject is greater than that in a subject to which the agent was not administered, thereby determining whether the agent promotes satellite cell (SC) proliferation.

Means of determining whether an agent, known to upregulate the sonic hedgehog pathway, can increase primary myoblast or satellite cell proliferation can include measuring Gli1 mRNA levels, as Gli1 is a Hedgehog downstream transcription factor, and therefore, a reporter of Hedgehog pathway activity (the Gli-1 gene sequence has GenBank accession number X07384, and the Gli-2 gene sequence has GenBank accession number AB007298). Said means of determination can include Cyclin D1, an established cell cycle regulator and

Gli transcriptional target. Said means of determination can also include Pax7, a transcription factor and marker for muscle satellite cells (SCs)(Seale, P. et al. (2000) Cell 102, 777-86).

Any of the above methods (e.g., screening methods) may be performed in vitro or in vivo. By way of example, said methods can be performed in isolated cells in culture, e.g., TM3 cells (a well-established Shh-sensitive cell line), as well as in any number of muscle or muscle precursor cells. Said muscle or muscle precursor cells can include primary myoblasts (PM), C2C12 myoblasts, and isolated satellite cells (SCs). By way of further example, said methods can be performed in animal models of myopathy, including but not limited to DMD^{MDX} mice, a model of muscular dystrophy. Muscle cells and tissue can be harvested from said animal models according to methods described herein.

This invention further provides a method for treating musculoskeletal disorders by administering to an afflicted subject a therapeutically effective amount of an agent known to agonize or otherwise upregulate the sonic hedgehog pathway and, determined to have the ability to increase cellular proliferation (e.g., of primary myoblasts and/or satellite cells (SCs)), wherein such ability is determined by a method comprising (i) administering the agent to a non-human subject, and (ii) determining whether the resulting cellular proliferation in the subject is greater than that in a subject to which the agent was not administered.

This invention further provides a method for inhibiting the onset of musculoskeletal disorders by administering to a subject in need thereof a prophylactically effective amount of an agent known to agonize or otherwise upregulate the sonic hedgehog pathway, and determined as having the ability to increase cellular proliferation (e.g., of primary myoblasts and/or satellite cells (SCs)), wherein such ability is determined by a method comprising (i) administering the agent to a non-human subject, and (ii) determining whether the resulting increase in cellular proliferation in the subject is greater than that in a subject to which the agent was not administered.

This invention further provides a composition comprising (a) a pharmaceutically acceptable carrier, and (b) an agent known to upregulate the sonic hedgehog pathway, and determined as having the ability to increase cellular proliferation (e.g., of primary myoblasts and/or satellite cells (SCs)), wherein such ability is determined by a method comprising (i) administering the agent to a non-human subject, and (ii) determining whether the resulting increase in cellular proliferation (e.g., of primary myoblasts and/or satellite cells (SCs)) in the subject is greater than that in a subject to which the agent was not administered.

This invention further provides an article of manufacture comprising a packaging material having therein an agent known to upregulate the sonic hedgehog pathway, and determined as having the ability to increase cellular proliferation (e.g., of primary myoblasts and/or satellite cells (SCs)), and a label indicating a use of the agent for inhibiting the onset of a musculoskeletal disorder in a subject, wherein such ability is determined by a method comprising (i) administering the agent to a non-human subject, and (ii) determining whether the resulting increase in cellular proliferation (e.g., of primary myoblasts and/or satellite cells (SCs)) in the subject is greater than that in a subject to which the agent was not administered.

This invention further provides an article of manufacture comprising a packaging material having therein an agent known to upregulate the sonic hedgehog pathway, and determined as having the ability to increase cellular proliferation (e.g., of primary myoblasts and/or satellite cells (SCs)), and a label indicating a use of the agent for treating a musculoskeletal disorder in a subject, wherein such ability is determined by a method comprising (i) administering the agent to a non-human subject, and (ii) determining whether the resulting increase in cellular proliferation (e.g., of primary myoblasts and/or satellite cells (SCs)) in the subject is greater than that in a subject to which the agent was not administered.

The methods of the present invention may be used to regulate proliferation of cells (e.g., of primary myoblasts and/or of satellite cells (SCs)) in vitro and/or in vivo, e.g., in the formation of new or regenerated muscle tissues. In another particular embodiment, contacting the cell with—or introducing into the cell—a compound of the invention (e.g., a compound of Formula I) results in promotion of cellular proliferation and recovery from musculoskeletal injury or disorder. Thus, another particular embodiment provides methods for agonizing the Hh pathway by employing compounds of the invention (e.g., a compound of Formula I) in an injured or diseased muscle cell.

### Hedgehog Pathway

Hedgehog (Hh) signaling was first identified in Drosophila as an important regulatory mechanism for embryonic pattern formation, or the process by which embryonic cells form ordered spatial arrangements of differentiated tissues. (Nusslein-Volhard et al. (1980) Nature 287, 795-801). Members of the Hedgehog family of signaling molecules mediate many important short- and long-range patterning processes during vertebrate development. Pattern formation is the activity by which embryonic cells form ordered spatial arrangements of differentiated tissues. The physical complexity of higher organisms arises during embryogenesis through the interplay of cell-intrinsic lineage and cell-extrinsic signaling. Inductive interactions are essential to embryonic patterning in vertebrate development from the earliest establishment of the body plan, to the patterning of the organ systems, to the generation of diverse cell types during tissue differentiation.

The vertebrate family of Hedgehog genes includes three members that exist in mammals, known as Desert (Dhh), Sonic (Shh) and Indian (Ihh) Hedgehogs, all of which encode secreted proteins. These various Hedgehog proteins consist of a signal peptide, a highly conserved N-terminal region, and a more divergent C-terminal domain. Biochemical studies have shown that autoproteolytic cleavage of the Hh precursor protein proceeds through an internal thioester intermediate which subsequently is cleaved in a nucleophilic substitution. It is likely that the nucleophile is a small lipophilic molecule which becomes covalently bound to the C-terminal end of the N-peptide, tethering it to the cell surface. The biological implications are profound. As a result of the tethering, a high local concentration of N-terminal Hedgehog peptide is generated on the surface of the Hedgehog producing cells. It is this N-terminal peptide which is both necessary and sufficient for short- and long-range Hedgehog signaling activities.

Downstream target genes of Hh signaling transcription include Wnts, TGFβ, and Ptc and Gli1, which are elements of the positive and negative regulatory feedback loop. Several cell-cycle and proliferation regulatory genes, such as c-myc, cyclin D and E are also among the target genes of Hh signaling.

Hedgehog genes encode secreted proteins, which undergo post-translational modifications, including autocatalytic cleavage and lipid modification (palmitoylation) at the N-terminus and cholesterol modification of the C-terminus. The lipid-modified N-terminal Hedgehog protein triggers the signaling activity of the protein pathway, and cell to cell communication is engendered by the dispatch of soluble Hedgehog protein from a signaling cell and receipt by a responding cell. In responding cells, the 12-pass transmembrane receptor Patched (Ptch) acts as negative regulator of Hh signaling and the 7-pass transmembrane protein Smoothened (Smo) acts as a positive regulator of Hh signaling. At resting state, free Ptch (i.e., unbound by Hh) substoichiometrically suppresses pathway activity induced by Smo (Taipale et al. (2002) Nature 418: 892); upon binding ligand Hh protein, however, repression of Smo is relieved, and the resulting signaling cascade leads to the activation and nuclear translocation of Gli transcription factors (Gli1, Gli2 and Gli3).

An inactive Hedgehog signaling pathway occurs where the transmembrane protein receptor Patched (Ptc) inhibits the stabilization, phosphorylation, and activity of Smoothened (Smo). The transcription factor Gli, a downstream component of Hh signaling, is prevented from entering the nucleus through interactions with cytoplasmic proteins, including Fused (Fu) and Suppressor of fused (Sufu). As a consequence, transcriptional activation of Hedgehog target genes is repressed. Activation of the pathway is initiated through binding of any of the three mammalian ligands (Dhh, Shh or Ihh) to Ptc.

Ligand binding by Hh alters the interaction of Smo and Ptc, reversing the repression of Smo, whereupon Smo moves from internal structures within the cell to the plasma membrane. The localization of Smo to the plasma membrane triggers activation of Hh pathway target genes in an Hh-independent manner. (Zhu et al. (2003) Genes Dev. 17(10):1240) The cascade activated by Smo leads to the translocation of the active form of the transcription factor Gli to the nucleus. The activation of Smo, through translocated nuclear Gli, activates Hh pathway target gene expression, including of Wnts, TGFIβ, and Ptc and Gli themselves.

This process can also occur in a native ligand-independent manner, such as through the administration of Shh agonizing compounds, recombinant Shh protein, or other Shh mimetics.

Hh signaling is known to regulate a diverse range of biological processes, such as cellular proliferation, differentiation, and organ formation in a tissue specific and dose dependent manner. In the development of neural tubes, Shh is expressed in the floorplate and directs the differentiation of specific subtypes of neurons, including motor and dopaminergic neurons. Hh regulates the proliferation of neuronal progenitor cells, such as cerebella granule cells and neural stem cells.

Hh signaling also plays important roles in normal tissue homeostasis and regeneration. The Hh pathway is activated after injury in such tissues as retina, bile duct, lung, bone and prostate in mouse models. Hh pathway is constantly active in hair follicles, bone marrow, and certain regions of the central nervous system (CNS), and benign prostate hyperplasia and blood vessel formation in wet macular degeneration require Hedgehog pathway activity.

Skeletal muscle contains resident stem cells, the most well-characterized being muscle satellite cells (SC), which provide a means for intrinsic regeneration of skeletal muscle tissue. However, in cases of injury, genetic disease, or aging, this regenerative capacity is reduced. Sonic hedgehog (Shh) signaling is critical for precursor cell expansion during embryonic muscle development and becomes reactivated in adults following muscle damage.

### Administration and Pharmaceutical Compositions:

The invention relates to the use of pharmaceutical compositions comprising compounds of Formula I in the therapeutic (and, in a broader aspect of the invention, prophylactic) treatment of musculoskeletal disorder(s).

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions.

The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). For example, synergistic effects can occur with immunomodulatory or anti-inflammatory substances or other anti-tumor therapeutic agents. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base.

Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions,” John Wiley And Sons, Inc., 1981.

### EXAMPLES

The present invention is further exemplified, but not limited, by the following representative examples, which are intended to illustrate the invention and are not to be construed as being limitations thereon. Unless otherwise indicated, said examples employ the following materials and methods:

Cells. TM3 and C2C12 cells were obtained from ATCC, propagated according to manufacturer's instructions, and maintained at subconfluence. Both primary myoblasts and isolated satellite cells were grown in Ham's F10 media supplemented with 20% Fetal calf serum and 1% Penicillin/Streptomycin without βFGF. All cells were plated on pre-coated collagen dishes (MatTek) or on plastic culture plates treated with rat tail collagen type I (BD).

RNA and qRT-PCR. For analysis of gene expression, cells were treated with vehicle (DMSO) or 10 nM Shh-Ag for 24 hours. Total RNA was isolated with RNEasy Miniprep Kit (Qiagen), DNAse treated (RQ1, Promega) and reverse transcribed (iScript, Biorad) according to manufacturers instructions. Quantitative PCR was performed using Taqman Gene Expression Assays (Applied Biosystems) in an ABI7500 Fast system as per manufacturer's instructions. Gene expression changes were calculated using the comparative ΔΔCT method.

Protein expression. For details of protein extraction and analysis, please see Supplementary Methods online. Antibodies used for Western are rabbit polyclonal antibodies against Gli1, total Akt, phosphorylated Akt-Ser473 (Cell Signaling) and Cyclin D1 (Abeam).

Myoblast and Satellite cell isolation. Primary mouse myoblasts were isolated from mouse hind-limb muscles as described herein. Muscle satellite cells were isolated from hind-limb muscles of 4-6 week old C57BL/6J mice. Muscles were dissected and finely chopped with razor blades in DMEM. Chopped muscles were placed in a 50 mL Falcon tube with 30 mL of dissociation solution (0.1% Collagenase D, 0.25% Trypsin in DMEM) and agitated at 37° for 20 minutes. Following dissociation, 500 μl of fetal calf serum was added to block Trypsin activity and samples were passed through a 37 μM filter. Solutions were diluted two-fold in DMEM, passed through a 70 μM filter, centrifuged at 1000 RPM for 15 minutes, and the subsequent pellet was re-suspended in 1% FCS/PBS. Syndecan 4 positive satellite cells were then isolated by fluorescent activated cell sorting (FACS) utilizing an ARIA-UV cell sorter (BD).

Mice. All mouse protocols were approved by the Animal Care and Use Committee of Novartis Institutes for BioMedical Research. Wild-type (C57BL/6J) and C57BL/10 DMD^{MDX} mice were obtained from Jackson Labs. All analyses were performed on male mice approximately 6-8 weeks of age.

Cardiotoxin-induced regeneration. Muscle regeneration was induced by injecting 40 μl (TA) or 100 μl (GA) of cardiotoxin (10 μM Naja atra; Sigma) into the respective tibialis anterior or gastrocnemius muscles of WT C57BL/6J mice.

Shh-Ag treatment. 20 μl (TA) or 50 μl (GA) of vehicle (10% DMSO/PBS) or 500 μM Shh-Ag was injected into the respective tibialis anterior or gastrocnemius muscles once per day for two consecutive days.

BrdU treatment. For treatment of isolated SCs, a 1:100 dilution of 5-bromodeoxyuridine (BrdU) labeling reagent (10 mg/mL; Invitrogen) was added to the culture media 24 hours following vehicle or Shh-Ag treatment. Cells were then incubated an additional 5 hours, fixed, probed for BrdU, mounted with Dapi containing medium (Vector Labs), and then imaged as described in Materials in Methods. Three random images from four replicate experiments were analyzed for total cell (Dapi) and BrdU+ cell number. For in vivo delivery, 100 μl of BrdU was intraperitoneally (IP) injected following the second vehicle or Shh-Ag intramuscular injection.

FACS analysis. The following antibodies were used in this study: APC-conjugated anti-CD45 (BD), PE-conjugated anti-Syndecan 4 (BD), and FITC-conjugated anti-BrdU (BioVision). Muscles from treated mice were dissected and dissociated as described. To sort satellite cells for further experimentation, APC-conjugated anti-syndecan 4 was utilized to positively select cells following ARIA (BD) mediated cell sorting. To analyze relevant BrdU expression, isolated cells were fixed in 1% paraformaldehyde in PBS for 15 minutes and stored in 70% ETOH at −20° C. Cells were then permeabilized in Tween 20 solution (0.2% in PBS; Sigma) for 10 mM, washed, and incubated with the described antibodies for 30 min before FACS analysis using a LSRII (BD).

Immunostaining. The following antibodies were used in this study: anti-laminin rabbit polyclonal (1:25; Sigma), anti-BrdU rat monoclonal (1:100; Abcam), and anti-Pax7 mouse monoclonal (1:25; DSHB, University of Iowa). Muscles were dissected and fixed in 10% Formalin/PBS overnight, processed for paraffin-embedding, and cut into 10 μM sections. Muscle sections were then blocked and stained with the aforementioned antibodies using a Discovery XT system (Ventana). Images were obtained using an ScanScope XT scanner and analyzed using Aperio ImageScope 6.25 software (Aperio Technologies).

Immunofluorescence. For immunofluorescent staining, the previous antibodies were utilized along with conjugated secondary antibodies against mouse, rat, and/or goat (Alexa Fluor 488/555, 1:400; Invitrogen). The sections were subsequently mounted in media containing DAPI (Vector Labs) and visualized using an AxioImager microscope (Zeiss).

Statistics. All values are expressed as mean±standard deviation (s.d.). To determine significance between two groups, vehicle vs. Shh-Ag treated, we made comparisons using an unpaired student t-test where a P<0.05 considered statistically significant.

Protein Expression. Cells were collected in lysis buffer (20 mM Tris, pH 7.5, 150 mM NaCl, 1 mM EDTA, 1 mM EGTA, 1% Nonidet NP-40, 1 mM β-glycerol phosphate, 1 mM Na₃VO₄, 1 μg/ml leupeptin, and 1 mM PMSF) and measured for protein concentration with a BCA protein assay kit (Pierce). Electrophoretic separation was performed utilizing 20-60 μg of protein on 4-12% mini-gels (Invitrogen). Proteins were transferred to a PVDF membrane, blocked in 5% blotto [5% powdered milk in TBST (Tris-buffered saline, 1% Tween 20)] for 1 hr followed by an overnight incubation at 4° C. with primary antibody. After overnight incubation, membranes were washed for 30 min in TBST and then probed with anti-rabbit antibody for 45 min at room temperature. After another 30 min of washing in TBST, the blots were developed with ECL. Once the appropriate image was captured, membranes were stained with Coomassie blue to verify equal loading in all lanes. Densitometric measurements were carried out using AlphaEase software (Alpha Innotech).

Myoblast isolation. Two to three week-old male C57/BL/6J pups were used for the experiments. The mice were euthanized using CO₂. Then the skin and fascia from the ankle joint up toward the hip were peeled to expose underlying muscles. The muscles (hamstring, quadriceps, tibialis anterior, extensor digitorum longus, gastrocnemius and soleus) from both legs were carefully removed, trying to any tendons and non-muscle tissues. The dissected muscles were placed in a sterile 10 cm² dish containing PBS and washed to remove hair and other debris. The muscles were then transferred to a sterile 35 mm² dish and 1.5 ml of a collagenase/dispase solution was added to digest the tissues. The tissues were mulched into a reasonably smooth pulp using sterile disposable followed by incubating at 37° C. for 12 min. They were then triturated using 5 ml plastic pipettes to homogenize the mixture. The incubation and triturating step was repeated once more.

The digested tissue mixture was further triturated 15 to 20 times with a 5 ml pipette prior to loading onto a 70 μm filter. The filtered mixture was centrifuged at 1000 rpm for 5 min. The cell pellet was resuspended in 3 ml of growth medium and plated on a 60 mm² non-collagen coated dish. The plates were incubated at 37° C. for 2 to 4 hr to allow the fibroblasts to attach while the majority of the myoblasts were still in suspension. The supernatant containing myoblasts was then removed from the non-collagen coated plates and seeded in collagen-coated plates. The cells were fed daily with the growth medium and split when the monolayers reached 70% confluency to enrich and expand myoblasts. A relatively pure population of myoblasts was obtained after three to four weeks of enrichment.

### Example 1

### In Vitro Determination of Shh-Ag Effects on Shh Signaling

To determine if Shh-Ag could activate Shh signaling in muscle cells, expression of the target and downstream functional gene, Gli1, was examined by quantitative PCR analysis in both a well-established Shh-sensitive cell line, TM3, along with three other murine muscle cell lines: primary myoblasts (PM), commercial C2C12 myoblasts, and isolated SCs. Gli1 gene expression, measured by real-time taqman assay and normalized to GAPDH levels, was compared in both vehicle (DMSO) and Shh-Ag treated (10 μM) cells, according to the methods and protocols described supra.

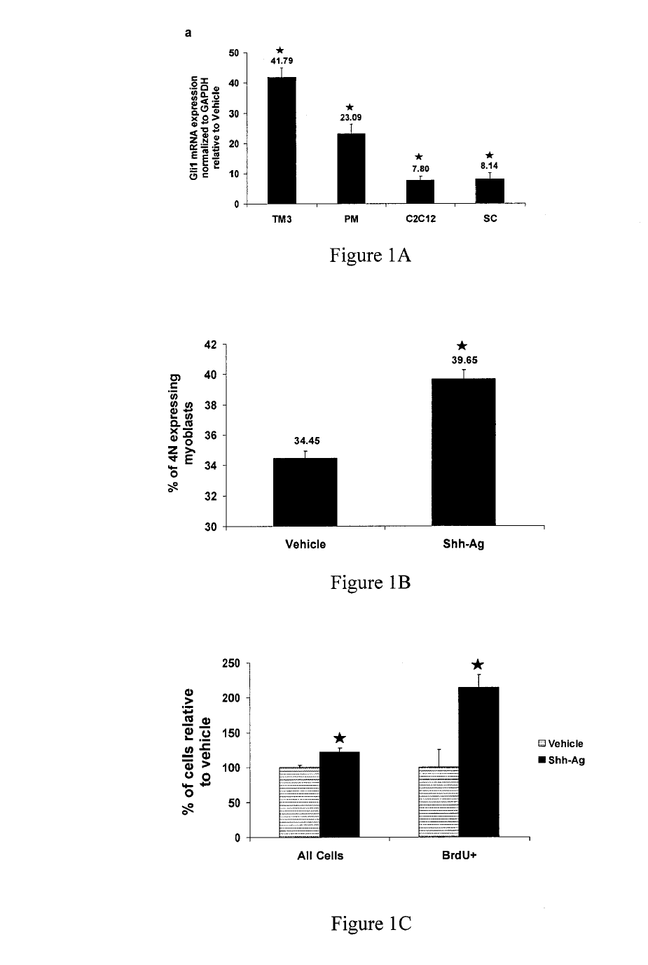

As seen in FIG. 1, 24 hours of Shh-Ag treatment (10 μM) robustly increased Gli1 mRNA levels in all cell lines examiner (i.e., over 40 fold in the TM3 cells, and over 20-fold in both myoblasts and myotubes, respectively). FIG. 1A shows 41.79±3.09 in TM3 cells; 23.09±3.21 in PM cells; 7.80±1.24 in C2C12 cells; and 8.14±2.01 in SCs (respective fold change relative to vehicle equaling one following normalization to GAPDH). Although baseline Gli1 expression levels were much lower in differentiated myotubes (see, e.g., FIG. 1C), this data is consistent with other cell types where Shh signaling is reduced with differentiation but suggests that myotubes retain the ability to respond to Shh activation. Shh-Ag treatment also had a similar effect on the commercially available C2C12 cell line.

After establishing that Shh-Ag treatment can induce Shh signaling, the effects of Shh-Ag on cell proliferation were examined through fluorescent activated cell sorting (FACS) analysis. Twenty-four hours of Shh-Ag treatment (10 nM) induced proliferation of primary myoblasts, demonstrated by an increased percentage of cells in G₂/M phase of the cell cycle upon propidium iodide (PI) staining and FACS analysis (see, e.g., FIG. 1B; 34.4±0.48 versus 39.6±0.61). Next, the mitogenic activity of isolated satellite cells following 48 hr of Shh-Ag treatment was assessed, and a significant increase in SC number, as well as a two fold increase in the percentage of SCs incorporating the proliferation marker BrdU, was found (see, e.g., FIG. 1C; 100±2.8 versus 121±5.8 and 100±24.7 versus 214±17.8; respectively).

To address the molecular mechanism for the observed Shh-Ag mediated effects on muscle cell proliferation, potential synergism with the phosphoinositide 3-kinase (PI3K)/Akt signaling pathway was examined. Insulin-like growth factor I (IGF-1) signaling through PI3K/Akt is a known skeletal muscle mitogen, and rShh-mediated induction of muscle cell proliferation has been shown to require Akt activation. (Elia, D. et al. (2007) Biochim Biophys Acta 1773, 1438-46)(Coolican, S. A., et al. (1997) J Biol Chem 272, 6653-62).

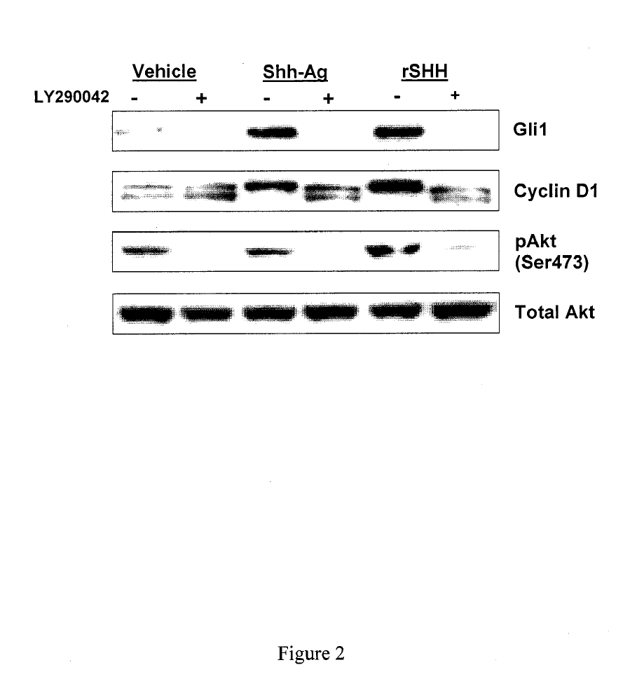

Co-treatment of Shh-Ag with IGF-1 did not significantly increase myoblast proliferation; however, treatment with LY294002, a potent inhibitor of the PI3K/Akt signaling pathway, completely blocked cell proliferation (see, e.g., FIG. 2A). LY294002 treatment also blocked both Shh-Ag and rShh-mediated induction of Gli1 protein levels (see, e.g., FIG. 2B), further supporting a role for Akt in the regulation of downstream Shh activation. Cyclin D1, an established cell cycle regulator and Gli transcriptional target, also showed elevated protein expression following Shh-Ag treatment (see, e.g., FIG. 2A). Additionally, a more than 50% increase in Cyclin D1 mRNA expression following Shh-Ag treatment in both myoblasts and isolated SCs was observed. These results are consistent with observations of synergistic co-stimulation of Cyclin D1 by Shh and other growth signaling pathways such as Epidermal Growth Factor. (Kasper, M. et al. (2006) Mol Cell Biol 26, 6283-98). Taken together, these results confirm that Shh-Ag treatment is dependent on the PI3K/Akt signaling pathway for induction of downstream targets and promotion of muscle precursor cell proliferation.

### Example 2

### Intramuscular Injection of Shh-Ag Induces Satellite Cell Proliferation In Vivo

Shh-Ag was tested in vivo by performing direct intramuscular (IM) injections of Shh-Ag into mouse skeletal muscle. Tibialis anterior (TA) or Gastrocnemius (GA) muscles of wild-type (WT) or DMD^{MDX} mice, a model of muscular dystrophy, were injected with 20 μl (TA) or 40 μl (GA) of either vehicle (10% DMSO/PBS) or Shh-Ag (500 μM) once per day for two consecutive days. Immediately following the second IM injection, the mice were also intraperitoneally (IP)-injected with 100 μl of BrdU. Twenty-four hours following the BrdU injection, the mice were sacrificed and their muscles were collected and prepared for FACS analysis (GA) or immunostaining (TA). For immunostaining, the muscle sections were probed with anti-laminin to mark the basal lamina, along with anti-BrdU to identify proliferating cells, and treated with hematoxylin dye to stain all nuclei.

While WT vehicle-injected muscle sections were negative for BrdU staining, Shh-Ag injected muscles displayed BrdU+ precursor cells. Interestingly, while DMD^{MDX} muscle displayed a high background of BrdU+ staining, Shh-Ag injections seemed to promote even greater mononuclear cell infiltration. To determine if resident proliferating cells (BrdU+) stimulated by Shh-Ag treatment are indeed satellite cells (SCs), we stained the sections for Pax7, a transcription factor and marker for muscle SCs. As such, Pax7+ SCs were appropriately located at the periphery of WT and DMD^{MDX} muscle. As expected, the continually-regenerating DMD^{MDX} muscle displayed a high number of Pax7+ SCs along with centrally-nucleated myofibers; however, some non-specific staining was also evident in areas of high cell damage.

Because co-visualization of nuclear expressed Pax7/BrdU is difficult, fluorescent co-staining was performed to determine if BrdU+ cells in Shh-Ag treated muscles are indeed Pax7+ SCs. In WT vehicle-injected muscle sections, all Pax7 expressing SCs were negative for BrdU, whereas Pax7+/BrdU+ double positive cells were easily seen in WT Shh-Ag injected muscles. In DMD^{MDX} muscle, most SCs were BrdU+ under normal untreated conditions, but Shh-Ag treatment greatly increased the number of Pax7−/BrdU+ cells. Taken together, these results demonstrate that in WT muscle, Shh-Ag injection directly activates SC proliferation, but in the continually-regenerating state of DMD^{MDX} muscle, the proliferative-response of Pax7-expressing SCs is already maximized.

Because of the unique pathological state of DMD^{MDX} muscle, the effect of Shh-Ag treatment on WT muscle regeneration was investigated, utilizing the well-established model of cardiotoxin-induced injury. (Cooper, R. N. et al. (1999) J Cell Sci 112, 2895) (Meeson, A. P. et al. (2004) Stem Cells 22, 1305-20) In response to a single cardiotoxin (ctx) injury, Shh-Ag treatment was found to significantly increase the number of BrdU+ cells at an early stage of regeneration. Furthermore, fluorescent co-staining of Pax7 and BrdU demonstrates that a larger percentage of Pax7 expressing SCs are Pax7+/BrdU+ double positive in Shh-Ag treated muscle compared to vehicle control.

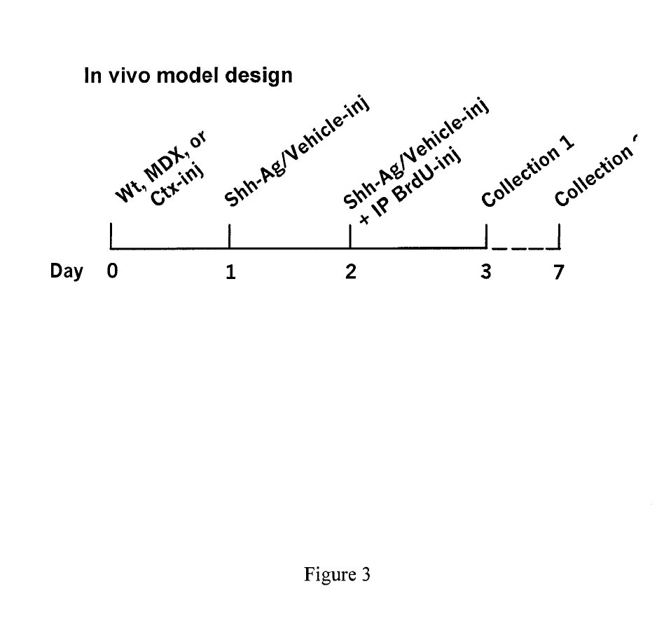

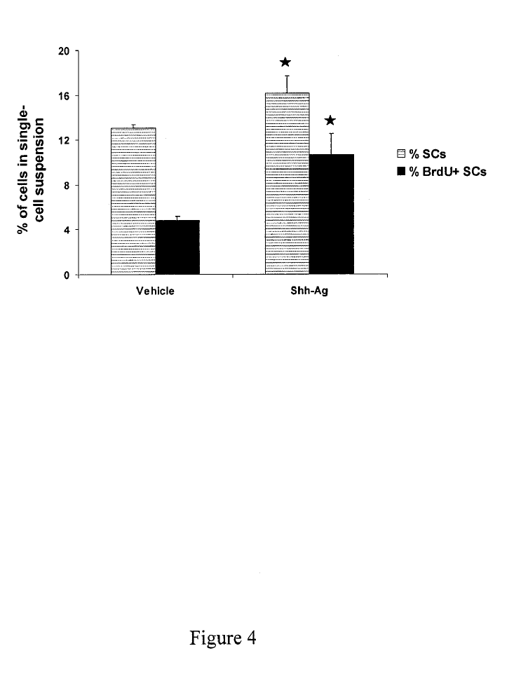

To quantify the induction of SC proliferation, ctx-injured and Shh-Ag treated muscle samples (GA) were collected and processed for FACS analysis. A single-cell muscle suspension was gated based on CD45− and Syndecan4+ expression, commonly used identifiers of muscle SCs, and the resultant population was then analyzed for intracellular BrdU localization (see, e.g., FIG. 4). Shh-Ag treatment only modestly increased the total percentage of SCs in the muscle samples, but more than doubled the percent of proliferating (BrdU+) SCs ((see FIG. 3); 13.06±0.31 versus 16.15±1.57 for total percentage and 4.82±0.33 versus 10.67±1.87 for percentage of BrdU+; respectively).

To determine if this Shh-Ag activation of early satellite cell proliferation results in a lasting improvement in muscle regeneration, muscles were treated as previously described and analyzed at a later timepoint (7 days) following ctx-injury. Longitudinal muscle sections co-stained with anti-laminin and BrdU demonstrate an increase in BrdU+, centrally-aligned nuclei in Shh-Ag treated muscles ((see FIG. 3)). Since skeletal muscle nuclei are post-mitotic, BrdU+ myonuclei represent proliferative precursors that have ultimately fused with and contributed to regenerating muscle fibers. Increases in BrdU+ myonuclei clearly demonstrate a functional impact of early SC activation following Shh-Ag treatment.

What is claimed is:

1. A method for treating musculoskeletal disorders comprising administering to an afflicted subject a therapeutically effective amount of an agonist of the Sonic Hedgehog (Shh) pathway.

2. The method of claim 1, wherein said Shh agonist comprises a compound of formula (I): 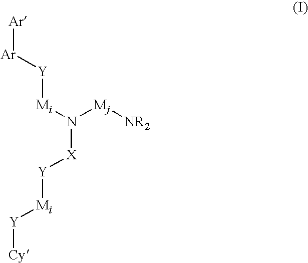
wherein, as valence and stability permit,
Ar and Ar′ independently represent substituted or unsubstituted aryl or heteroaryl rings;
Y, independently for each occurrence, is absent or represents —N(R)—, —O—, —S—, or —Se—;
X is selected from —C(═O)—, —C(═S)—, —S(O₂)—, —S(O)—, —P(═O)(OR)—, and a methylene group optionally substituted with 1-2 groups such as lower alkyl, alkenyl, or alkynyl groups;
M represents, independently for each occurrence, a substituted or un-substituted methylene group, such as —CH2—, —CHF—, —CHOH—, —CH(Me)—, —C(═O)—, etc., or two M taken together represent substituted or unsubstituted ethene or ethyne, wherein some or all occurrences of M in Mⱼ form all or part of a cyclic structure;
R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;
Cy′ represents a substituted or unsubstituted aryl, heterocyclyl, heteroaryl, or cycloalkyl, including polycyclic groups;
j represents, independently for each occurrence, an integer from 0 to 10, preferably from 2 to 7; and
i represents, independently for each occurrence, an integer from 0 to 5, preferably from 0 to 2.

3. The method of claim 2, wherein said Shh agonist is Shh-Ag.

4. The method of claim 1, wherein said musculoskeletal disorder is a muscular dystrophy.

5. The method of claim 1, wherein said musculoskeletal disorder is a myopathy.

6. A method for diagnosing musculoskeletal disorders comprising administering to an afflicted subject a therapeutically effective amount of an agonist of the Sonic Hedgehog (Shh) pathway.

7. The method of claim 6, wherein said Shh agonist comprises a compound of formula (I): 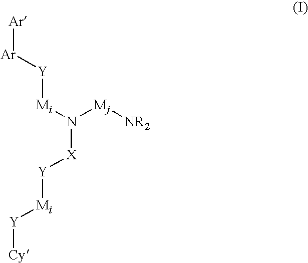
wherein, as valence and stability permit,
Ar and Ar′ independently represent substituted or unsubstituted aryl or heteroaryl rings;
Y, independently for each occurrence, is absent or represents —N(R)—, —O—, —S—, or —Se—;
X is selected from—C(═O)—, —C(═S)—, —S(O₂)—, —S(O)—, —C(═NCN)—, —P(═O)(OR)—, and a methylene group optionally substituted with 1-2 groups such as lower alkyl, alkenyl, or alkynyl groups;
M represents, independently for each occurrence, a substituted or un-substituted methylene group, such as —CH2-, —CHF—, —CHOH—, —CH(Me)—, —C(═O)—, etc., or two M taken together represent substituted or unsubstituted ethene or ethyne, wherein some or all occurrences of M in Mⱼ form all or part of a cyclic structure;
R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;
Cy′ represents a substituted or unsubstituted aryl, heterocyclyl, heteroaryl, or cycloalkyl, including polycyclic groups;
j represents, independently for each occurrence, an integer from 0 to 10, preferably from 2 to 7; and
i represents, independently for each occurrence, an integer from 0 to 5, preferably from 0 to 2.

8. The method of claim 7, wherein said Shh agonist is Shh-Ag.

9. The method of claim 6, wherein said musculoskeletal disorder is a muscular dystrophy.

10. The method of claim 6, wherein said musculoskeletal disorder is a myopathy.

11. A method for determining whether a test agent increases primary myoblast proliferation comprising: (i) administering the test agent known to upregulate the Sonic Hedgehog (Shh) pathway to a non-human subject; and (ii) determining whether the resulting primary myoblast proliferation in the subject is greater than that in a subject to which the agent was not administered, thereby determining whether the agent increases primary myoblast proliferation.
